(19) United States
(12) Patent Application Publication
(10) Pub. No.: US 2010/0093864 A1
(43) Pub. Date: Apr. 15, 2010

(54) ANTI-INFECTIVE THIOUREA COMPOUNDS

(75) Inventors: Simon Feldbaek Nielsen, Herlev (DK); Arsalan Kharazmi, Kobenhavn (DK); Mogens Larsen, Smorum (DK)

Correspondence Address: Muncy, Geissler, Olds & Lowe, PLLC, P.O. BOX 1364, FAIRFAX, VA, 22038-1364, US

(73) Assignee: LICA PHARMACEUTICALS, Kobenhavn O (DK)

(21) Appl. No.: 12/445,434

(22) PCT Filed: Oct. 12, 2007
(86) PCT No.: PCT/EP07/60894

§ 371 Date: Jul. 16, 2009

Related U.S. Application Data
(60) Provisional application No. 60/851,637, filed on Oct. 13, 2006.

(30) Foreign Application Priority Data
Oct. 13, 2006 (EP) 06021551.4

(51) Int. Cl. A61K 31/17; C07C 335/16; A61P 31/04; A61P 31/10

(52) U.S. Cl. 514/587; 564/29; 564/28; 514/585

(57) ABSTRACT

The present application discloses thiourea compounds for use in the treatment of infections. The thiourea compounds have formula (I) wherein X¹ and X² each designate one or more substituents; and Z is —(CHR)—, —(CHR)₂—, —O—(CHR)₂—, —NH—(CHR)₂—, or —S—(CHR)₂—; including salts thereof.

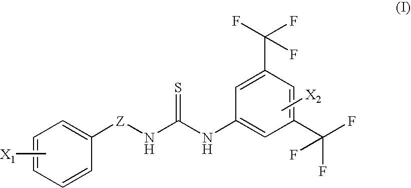

### FIELD OF THE INVENTION

The present invention relates to thiourea compounds and in particular to the use of such compounds in the treatment of infections.

### BACKGROUND OF THE INVENTION

The spread of antimicrobial resistance determinants particular among nosocomial bacterial pathogens is an increasing problem. Such resistant pathogens include Staphylococcus aureus resistant to methicillin and thus to all β-lactam-antibiotics and Enterococci resistant to vancomycin (VRE). Such resistant bacteria pose a significant therapeutic challenge and bacterial strains resistant to all currently available antimicrobials are emerging. Furthermore, bacterial species intrinsically resistant to commonly employed antimicrobials are being recognized as important opportunistic pathogens in the setting of long-term immuno-compromized patients. An example of this is Stenotrophomonas maltophilia which possesses a β-lactamase rendering the bacteria intrinsically resistant to carbapenems. As cross-resistance within a given class of antibiotics often occurs, the development of new classes of antibiotics is a necessity to counter the emerging threat of bacterial resistance.

Thus, there is a need for novel compound classes with improved therapeutic activity against bacteria and other microorganisms, such as fungi and mycoplasma.

Thiourea compounds of the type defined herein are sporadically known from WO 2000/034268 A1, WO 2000/034269 A1, WO 2000/034261 A2, WO 2000/034260 A2, WO 2000/034258 A2, WO 2000/034238 A1, WO 2000/034237 A2, (all relating to thiourea derivatives useful as inhibitors of herpes viruses), WO 1999/07672 A1 (relating to potassium channel openers), FR 1511325 (relating to thiourea derivatives useful against mollusks and snails).

NL 6516437 and U.S. Pat. No. 3,660,484 discloses thiourea compounds as, e.g., bactericidal and fungicidal agents.

### DESCRIPTION OF THE INVENTION

The present inventors have found that the thiourea compounds defined herein exhibit properties which are very useful for combating infections in mammalian species.

Hence, the present invention i.a. provides the use of a thiourea compound for the preparation of a pharmaceutical composition for the treatment of an infection, said compound having the Formula I

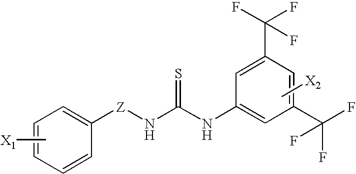

wherein
X¹ designate a substituent present 0-5 times on the respective benzene ring and X² designate a substituent present 0-3 times on the respective benzene ring, each X¹ and X² independently being selected from the group consisting of optionally substituted C₁₋₁₂-alkyl, optionally substituted C₂₋₁₂-alkenyl, optionally substituted C₄₋₁₂-alkadienyl, optionally substituted C₆₋₁₂-alkatrienyl, optionally substituted C₂₋₁₂-alkynyl, hydroxy, optionally substituted C₁₋₁₂-alkoxy, optionally substituted C₂₋₁₂-alkenyloxy, carboxy, optionally substituted C₁₋₁₂-alkoxycarbonyl, optionally substituted C₁₋₁₂-alkylcarbonyl, formyl, C₁₋₆-alkylsulphonylamino, optionally substituted aryl, optionally substituted aryloxy-carbonyl, optionally substituted aryloxy, optionally substituted arylcarbonyl, optionally substituted arylamino, arylsulphonylamino, optionally substituted heteroaryl, optionally substituted heteroaryloxycarbonyl, optionally substituted heteroaryloxy, optionally substituted heteroarylcarbonyl, optionally substituted heteroarylamino, heteroarylsulphonylamino, optionally substituted heterocyclyl, optionally substituted heterocyclyloxycarbonyl, optionally substituted heterocyclyloxy, optionally substituted heterocyclylcarbonyl, optionally substituted heterocyclylamino, heterocyclylsulphonylamino, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, amino-C₁₋₆-alkyl-carbonylamino, mono- and di(C₁₋₆-alkyl)amino-C₁₋₆-alkyl-carbonylamino, cyano, guanidino, carbamido, C₁₋₆-alkanoyloxy, C₁₋₆-alkylsulphonyl, C₁₋₆-alkylsulphinyl, C₁₋₆-alkylsulphonyloxy, aminosulfonyl, mono- and di(C₁₋₆-alkyl)aminosulfonyl, nitro, optionally substituted C₁₋₆-alkylthio, and halogen, where any nitrogen-bound C₁₋₆-alkyl is optionally substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino; or two X¹ substituents may together form an —OCH(R¹)O— group wherein R¹ is selected from the group consisting of hydrogen, C₁₋₆-alkyl and phenyl;
wherein Z is selected from the group consisting of —(CHR)—, —(CHR)₂—, —O—(CHR)₂—, —NH—(CHR)₂—, and —S—(CHR)₂—, wherein each R individually is selected from the group consisting of hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, optionally substituted C₁₋₆-alkylcarbonyl, optionally substituted aryl, optionally substituted aryloxy, optionally substituted arylcarbonyl, optionally substituted heteroaryl, optionally substituted heteroaryloxy, optionally substituted heteroarylcarbonyl, optionally substituted heterocyclyl, optionally substituted heterocyclyloxy, optionally substituted heterocyclylcarbonyl;
including salts thereof.

In the currently most preferred embodiments, R is selected from the group consisting of hydrogen and C₁₋₆-alkyl.

A great deal of variety is allowed for the substituents X¹ and X². Most often, however, X¹ and X² independently designates 0-4 substituents, where such optional substituents independently are selected from optionally substituted C₁₋₁₂-alkyl, hydroxy, optionally substituted C₁₋₁₂-alkoxy, optionally substituted C₂₋₁₂-alkenyloxy, carboxy, optionally substituted C₁₋₁₂-alkylcarbonyl, formyl, C₁₋₆-alkylsulphonylamino, optionally substituted aryl, optionally substituted aryloxy-carbonyl, optionally substituted aryloxy, optionally substituted arylcarbonyl, optionally substituted arylamino, arylsulphonylamino, optionally substituted heteroaryl, optionally substituted heteroarylamino, optionally substituted heteroarylcarbonyl, optionally substituted heteroaryloxy, heteroarylsulphonylamino, optionally substituted heterocyclyl, optionally substituted heterocyclyloxy, optionally substituted heterocyclylamino, amino, mono- and di(C₁₋₆-alkyl)amino, carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, amino-C₁₋₆-alkyl-carbonylamino, mono- and di(C₁₋₆-alkyl)amino-C₁₋₆-alkyl-carbonylamino, guanidino, carbamido, C₁₋₆-alkylsulphonyl, C₁₋₆-alkylsulphinyl, C₁₋₆-alkylsulphonyloxy, optionally substituted C₁₋₆-alkylthio, aminosulfonyl, mono- and di(C₁₋₆-alkyl)aminosulfonyl, and halogen, where any nitrogen-bound C₁₋₆-alkyl may be substituted with a substituent selected from the group consisting of hydroxy, C₁₋₆-alkoxy, and halogen.

More particular, X¹ and X² independently designate 0-3 substituents, such optional substituents independently being selected from optionally substituted C₁₋₆-alkyl, optionally substituted C₁₋₆-alkoxy, optionally substituted C₁₋₆-alkylcarbonyl, optionally substituted aryl, optionally substituted aryloxy, optionally substituted arylamino, optionally substituted heteroaryl, optionally substituted heteroarylamino, mono- and di(C₁₋₆-alkyl)amino, C₁₋₆-alkylcarbonylamino, optionally substituted C₁₋₆-alkylthio, optionally substituted heterocyclyl, optionally substituted heterocyclyloxy, optionally substituted heterocyclylamino and halogen, where any nitrogen-bound C₁₋₆-alkyl may be substituted with a substituent selected from the group consisting of hydroxy, C₁₋₆-alkoxy, and halogen.

In the currently most preferred embodiment, X¹ represents at least one substituent selected from C₁₋₆-alkyl, C₁₋₆-alkoxy, C₁₋₆-alkylcarbonyl, optionally substituted aryl, optionally substituted aryloxy, optionally substituted arylamino, optionally substituted heteroaryl, optionally substituted heteroarylamino, mono- and di(C₁₋₆-alkyl)amino, C₁₋₆-alkylcarbonylamino, optionally substituted C₁₋₆-alkylthio, optionally substituted heterocyclyl, optionally substituted heterocyclyloxy, optionally substituted heterocyclylamino, and halogen; in particular, X¹ represents 1-3 substituents selected from C₁₋₆-alkyl, C₁₋₆-alkoxy, and halogen. Even more preferred are the embodiments, wherein X¹ at least one halogen substituent.

On the other hand, it is currently preferred that no X² substituents are present.

It appears that the length of the group Z is not particularly critical, although it is currently preferred that Z is selected from the group consisting of —(CHR)₂— and —O—(CHR)₂—.

In one preferred embodiment, Z is —(CHR)— where R is selected from hydrogen and C₁₋₆-alkyl, preferably hydrogen and methyl. In another preferred embodiment, Z is —(CHR)₂—, where R is selected from hydrogen and C₁₋₆-alkyl, preferably hydrogen and methyl. In a further preferred embodiment Z is —O—(CHR)₂—, where R is selected from hydrogen and C₁₋₆-alkyl, preferably hydrogen and methyl. In a still further preferred embodiment Z is —S—(CHR)₂—, where R is selected from hydrogen and C₁₋₆-alkyl, preferably hydrogen and methyl. In a still further preferred embodiment Z is —NH—(CHR)₂—, where R is selected from hydrogen and C₁₋₆-alkyl, preferably hydrogen and methyl.

Some illustrative, but currently very interesting, examples of thiourea compounds Formula I are:

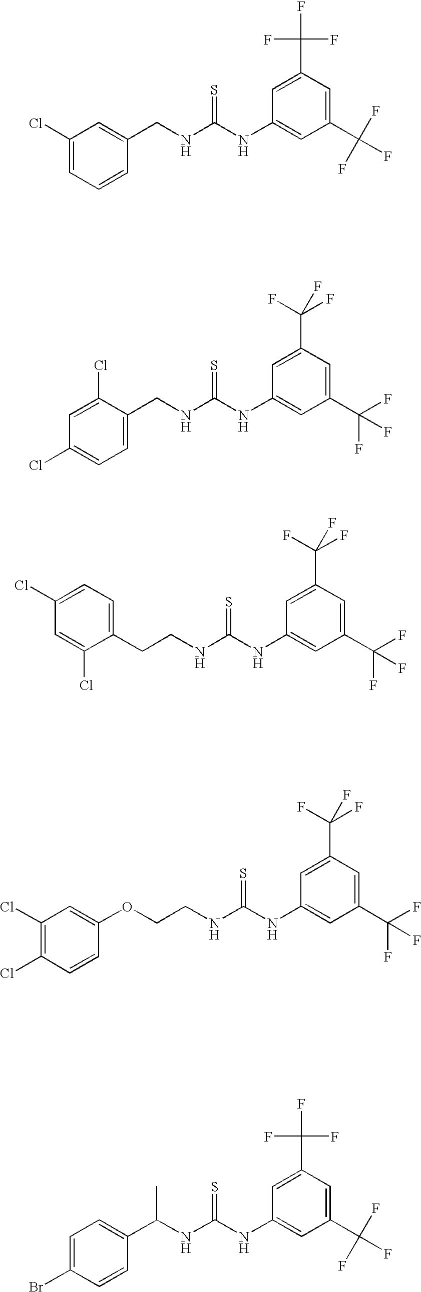

As is evident from the formulae defined herein and the definitions associated therewith, certain compounds of the present invention may be chiral. Moreover, the possible presence of multiple stereogenic atoms provides for the existence of diastereomeric forms of some of the compounds. The invention is intended to include all stereoisomers, including optical isomers, and mixtures thereof, as well as pure, partially enriched, or, where relevant, racemic forms.

### DEFINITIONS

In the present context, the term “infection” is intended to mean the pathological state resulting from the invasion of the body by pathogenic microorganisms. Hence, the term “infection” does not include the presence of pathogenic microorganisms on the exterior surface of the body.

In the present context, the term “anti-bacteral” is intended to describe an antimicrobial activity of a test compound, characterized by the reduction of viable bacteria (bacterial kill) during incubation with the test compound, as evidenced in the killing curve determination by a reduction of colony forming units (CFU) during incubation time.

In the present context, the term “C₁₋₁₂-alkyl” is intended to mean a linear, cyclic or branched hydrocarbon group having 1 to 12 carbon atoms, such as methyl, ethyl, propyl, iso-propyl, cyclopropyl, butyl, tert-butyl, iso-butyl, cyclobutyl, pentyl, cyclopentyl, hexyl, cyclohexyl, etc. Analogously, the term “C₁₋₆-alkyl” is intended to mean a linear, cyclic or branched hydrocarbon group having 1 to 6 carbon atoms, such as methyl, ethyl, propyl, iso-propyl, pentyl, cyclopentyl, hexyl, cyclohexyl, and the term “C₁₋₄-alkyl” is intended to cover linear, cyclic or branched hydrocarbon groups having 1 to 4 carbon atoms, e.g. methyl, ethyl, propyl, iso-propyl, cyclopropyl, butyl, iso-butyl, tert-butyl, cyclobutyl.

Whenever the term “C₁₋₁₂-alkyl” is used herein, it should be understood that a particularly interesting embodiment thereof is “C₁₋₆-alkyl”.

Similarly, the terms “C₂₋₁₂-alkenyl”, “C₄₋₁₂-alkadienyl”, and “C₆₋₁₂-alkatrienyl” are intended to cover linear, cyclic or branched hydrocarbon groups having 2 to 12, 4 to 12, and 6 to 12, carbon atoms, respectively, and comprising one, two, and three unsaturated bonds, respectively. Examples of alkenyl groups are vinyl, allyl, butenyl, pentenyl, hexenyl, heptenyl, octenyl, heptadecaenyl. Examples of alkadienyl groups are butadienyl, pentadienyl, hexadienyl, heptadienyl, heptadecadienyl. Examples of alkatrienyl groups are hexatrienyl, heptatrienyl, octatrienyl, and heptadecatrienyl. Preferred examples of alkenyl are vinyl, allyl, butenyl, especially allyl.

Similarly, the term “C₂₋₁₂-alkynyl” is intended to mean a linear or branched hydrocarbon group having 2 to 12 carbon atoms and comprising a triple bond. Examples hereof are ethynyl, propynyl, butynyl, octynyl, and dodecaynyl. Whenever the terms “C₂₋₁₂-alkenyl”, “C₄₋₁₂-alkadienyl”, “C₆₋₁₂-alkatrienyl”, and “C₂₋₁₂-alkynyl” are used herein, it should be understood that a particularly interesting embodiment thereof are the variants having up to six carbon atoms.

In the present context, i.e. in connection with the terms “alkyl”, “alkenyl”, “alkadienyl”, “alkatrienyl”, and “alkynyl”, the term “optionally substituted” is intended to mean that the group in question may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxycarbonyl, aryloxy, arylamino, arylcarbonyl, heteroaryl, heteroarylamino, heteroaryloxycarbonyl, heteroaryloxy, heteroarylcarbonyl, amino, mono- and di(C₁₋₆-alkyl)amino, carbamoyl, mono- and di(C₁₋₆-alkyl)-aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyl-oxy, nitro, C₁₋₆-alkylthio, halogen, where any aryl and heteroaryl may be substituted as specifically described below for “optionally substituted aryl and heteroaryl”, and any alkyl, alkoxy, and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Preferably, the substituents are selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkylcarbonyl, formyl, aryl, aryloxy, arylamino, arylcarbonyl, heteroaryl, heteroarylamino, heteroaryloxy, heteroarylcarbonyl, amino, mono- and di(C₁₋₆-alkyl)amino; carbamoyl, mono- and di(C₁₋₆-alkyl)-aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, guanidino, carbamido, C₁₋₆-alkyl-sulphonyl-amino, C₁₋₆-alkyl-sulphonyl, C₁₋₆-alkyl-sulphinyl, C₁₋₆-alkylthio, halogen, where any aryl and heteroaryl may be substituted as specifically described below for “optionally substituted aryl and heteroaryl”.

Especially preferred examples are hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, and guanidino.

The terms “optionally substituted C₁₋₁₂-alkoxy” and “optionally substituted C₁₋₆-alkoxy” are intended to mean that the alkoxy groups may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxycarbonyl, aryloxy, arylcarbonyl, heteroaryl, heteroaryloxycarbonyl, heteroaryloxy, heteroarylcarbonyl, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, halogen, where any aryl and heteroaryl may be substituted as specifically described below for “optionally substituted aryl and heteroaryl.

Especially preferred examples of “optionally substituted C₁₋₁₂-alkoxy” and “optionally substituted C₁₋₆-alkoxy” groups are unsubstituted such groups as well as those carrying one or two substituents selected from hydroxy, C₁₋₆-alkyl, C₁₋₆-alkoxy, C₂₋₆-alkenyloxy, carboxy, halogen, or C₁₋₆-alkylthio.

“Halogen” includes fluoro, chloro, bromo, and iodo.

In the present context, the term “aryl” is intended to mean a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

The term “heteroaryl” is intended to mean a fully or partially aromatic carbocyclic ring or ring system where one or more of the carbon atoms have been replaced with heteroatoms, e.g. nitrogen (═N— or NH—), sulphur, and/or oxygen atoms. Examples of such heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, triazinyl, coumaryl, furyl, thienyl, quinolyl, benzothiazolyl, benzotriazolyl, benzodiazolyl, benzooxozolyl, phthalazinyl, phthalanyl, triazolyl, tetrazolyl, isoquinolyl, acridinyl, carbazolyl, dibenzazepinyl, indolyl, benzopyrazolyl, phenoxazonyl. Particularly interesting heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, furyl, thienyl, quinolyl, triazolyl, tetrazolyl, isoquinolyl, indolyl in particular pyrrolyl, imidazolyl, pyridinyl, pyrimidinyl, thienyl, quinolyl, tetrazolyl, and isoquinolyl.

The term “heterocyclyl” is intended to mean a non-aromatic carbocyclic ring or ring system where one or more of the carbon atoms have been replaced with heteroatoms, e.g. nitrogen (═N— or —NH—), sulphur, and/or oxygen atoms. Examples of such heterocyclyl groups are imidazolidine, piperazine, hexahydropyridazine, hexahydropyrimidine, diazepane, diazocane, pyrrolidine, piperidine, azepane, azocane, aziridine, azirine, azetidine, pyrroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane, oxathiepane. The most interesting examples are imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular imidazolidine, piperazine, hexahydropyridazine, hexahydropyrimidine, diazepane, pyrrolidine, piperidine, azepane, oxazinane (morpholine), and thiazinane.

In the present context, i.e. in connection with the terms “aryl”, “heteroaryl”, and heterocyclyl, the term “optionally substituted” is intended to mean that the group in question may be substituted one or several times, preferably 1-5 times, in particular 1-3 times) with group(s) selected from hydroxy (which when present in an enol system may be represented in the tautomeric keto form), C₁₋₆-alkyl, C₁₋₆-alkoxy, C₂₋₆-alkenyloxy, oxo (which may be represented in the tautomeric enol form), carboxy, C₁₋₆-alkoxycarbonyl, C₁₋₆-alkylcarbonyl, formyl, aryl, aryloxy, arylamino, aryloxycarbonyl, arylcarbonyl, heteroaryl, heteroarylamino, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkyl-sulphonyl-amino, aryl-sulphonyl-amino, heteroaryl-sulphonyl-amino, C₁₋₆-alkyl-sulphonyl, C₁₋₆-alkyl-sulphinyl, C₁₋₆-alkylsulphonyloxy, nitro, sulphanyl, amino, amino-sulfonyl, mono- and di(C₁₋₆-alkyl)amino-sulfonyl, dihalogen-C₁₋₄-alkyl, trihalogen-C₁₋₄-alkyl, halogen, where aryl and heteroaryl representing substituents may be substituted 1-3 times with C₁₋₄-alkyl, C₁₋₄-alkoxy, nitro, cyano, amino or halogen, and any alkyl, alkoxy, and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Preferably, the substituents are selected from hydroxy, C₁₋₆-alkyl, C₁₋₆-alkoxy, oxo (which may be represented in the tautomeric enol form), carboxy, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocarbonyl, C₁₋₆-alkylcarbonylamino, guanidino, carbamido, C₁₋₆-alkyl-sulphonyl-amino, aryl-sulphonyl-amino, heteroaryl-sulphonyl-amino, C₁₋₆-alkyl-sulphonyl, C₁₋₆-alkyl-sulphinyl, C₁₋₆-alkylsulphonyloxy, sulphanyl, amino, amino-sulfonyl, mono- and di(C₁₋₆-alkyl)amino-sulfonyl or halogen, where any alkyl, alkoxy and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino. Especially preferred examples are C₁₋₆-alkyl, C₁₋₆-alkoxy, amino, mono- and di(C₁₋₆-alkyl)amino, sulphanyl, carboxy or halogen, where any alkyl, alkoxy and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkyl-carbonylamino, halogen, C₁₋₆-alkylthio, C₁₋₆-alkyl-sulphonyl-amino, or guanidino. In the present context the term “nitrogen-containing heterocyclic ring” is intended to mean heterocyclic ring or ring system in which at least one nitrogen atom is present. Such a nitrogen is, with reference to the general formula I (substituents A, B, and C), carrying the substituents R₁ and R₂. The “nitrogen-containing heterocyclic ring” may further comprise additional heteroatoms, e.g. nitrogen (═N— or —N—), sulphur, and/or oxygen atoms. Examples of such rings are aromatic rings such as pyridine, pyridazine, pyrimidine, pyrazine, triazine, thiophene, oxazole, isoxazole, thiazole, isothiazole, pyrrole, imidazole, pyrazole, tetrazole, quinoline, benzothiazole, benzotriazole, benzodiazole, benzoxazole, triazole, isoquinoline, indole, benzopyrazole, thiadiazole, and oxadiazole. The most interesting examples of aromatic rings are pyridine, pyridazine, pyrimidine, pyrazine, thiophene, tetrazole, oxazole, isoxazole, thiazole, isothiazole, pyrrole, imidazole, pyrazole, quinoline, triazole, isoquinoline, and indole, in particular pyridine, thiophene, imidazole, quinoline, isoquinoline, indole, and tetrazole.

Other examples of such rings are non-aromatic rings such as imidazolidine, piperazine, hexahydropyridazine, hexahydropyrimidine, diazepane, diazocane, pyrrolidine, piperidine, azepane, azocane, aziridine, azirine, azetidine, pyrroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, and thiazetane. The most interesting examples of non-aromatic rings are imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular imidazolidine, piperazine, hexahydropyridazine, hexahydropyrimidine, diazepane, pyrrolidine, piperidine, azepane, oxazinane (morpholine), and thiazinane.

In the present context, i.e. in connection with the term “nitrogen-containing heterocyclic ring”, the term “optionally substituted” is intended to mean that the group in question may be substituted one or several times, preferably 1-5 times, in particular 1-3 times) with group(s) selected from the same substituents as defined above for “optionally substituted aryl”.

This being said, it should furthermore be understood that the compounds defined herein include salts thereof, of which pharmaceutically acceptable salts are of course especially relevant for the therapeutic applications. Salts include acid addition salts and basic salts. Examples of acid addition salts are hydrochloride salts, fumarate, oxalate, etc. Examples of basic salts are salts where the (remaining) counter ion is selected from alkali metals, such as sodium and potassium, alkaline earth metals, such as calcium salts, potassium salts, and ammonium ions (+N(R′)₄), where the R′s independently designate optionally substituted C₁₋₆-alkyl, optionally substituted C₂₋₆-alkenyl, optionally substituted aryl, or optionally substituted heteroaryl). Pharmaceutically acceptable salts are, e.g., those described in Remington's—The Science and Practice of Pharmacy, 20th Ed. Alfonso R. Gennaro (Ed.), Lippincott, Williams & Wilkins; ISBN: 0683306472, 2000, and in Encyclopedia of Pharmaceutical Technology. However, generally preferred salt forming agents for application in the present invention are organic dicarboxylic acids such as oxalic, fumaric, and maleic acid, and the like.

### Preparation of Compounds

The thiourea compounds defined herein may be produced by methods known per se for the preparation of thiourea and urea compounds or methods which are analogous to such methods. Examples of excellent methods for preparing thiourea compounds are giving in the following

The coupling of an amine and a phenyl-isothiocyanate giving a thiourea compound is a standard reaction common to all skilled chemists. The reaction can be performed in various solvents. Further examples of preparation are described in reference books such as “Advanced Organic Chemistry” by Jerry March, 5^{th} ed. (e.g. p 1191). The reaction is illustrated for 3,5-ditrifluoromethyl-phenyl-isothiocyanate:

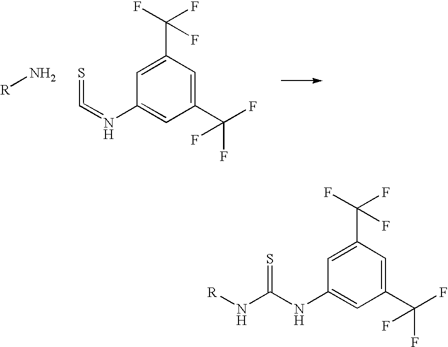

The corresponding phenyl-isothiocyanates (as illustrated above or including the X² substituent) can be obtained from the corresponding amines by methods known in the art.

### Therapeutic Uses

The present inventors have found that that the thiourea compounds has interesting properties which renders the compounds useful for combating infections in mammalian bodies, e.g. bacterial infections, fungal infections, mycoplasmic infections, and other infections caused by microorganisms (see the Examples section). It is of course possible that the compounds also have other interesting properties to be utilised in the medical field.

Hence as mentioned above, the thiourea compounds defined above are useful for the treatment of an infection.

Most typical, the infection is associated with bacteria, fungi, mycoplasma or other microorganisms.

In one aspect, the thiourea compound may be used for the treatment of bacterial infections in a mammal in need thereof. Such bacterial infection may be associated with common Gram-positive and/or Gram-negative pathogenes or with microaerophilic or anaerobic bacteria. As a particularly relevant example of bacteria against which thiourea compounds demonstrate an effect can be mentioned antibiotic-sensitive or -resistant strains of S. aureus and/or E. faecium. Other examples include community acquired and nosocomial respiratory infections, including S. pneumoniae, S. pyogenes and members of Enterobacteriaceae (e.g. E. coli), microaerophilic bacteria associated with gastric disease (e.g. Helicobacter pylori) or pathogenic anaerobic bacteria (e.g. Bacteroides fragilis and Clostridium species). The Examples section illustrates the anti-bacterial properties the compounds of the present invention. Corresponding in vivo results provide proof of the usefulness of the compounds.

In one embodiment, wherein the bacteria are selected from antibiotic-sensitive and -resistant strains of S. aureus. In another embodiment, the bacteria are a member of Enterobacteriaceae, e.g. E. coli.

In a still further embodiment, infection is associated with fungi or the infection is associated with mycoplasma.

The present invention also provides a method for treating infections (in particular the infections described above, such as bacterial infections) in a mammal comprising administration of a compound of the general formula I to a subject in need therefor.

Hence, the present invention also provides a method of treating a mammal suffering from an infection, said method comprising the step of administering a therapeutically effective amount of a thiourea compound of Formula I as defined herein to said mammal.

As above, the infection is most typical associated with bacteria, fungi, mycoplasma or other microorganisms.

It is furthermore envisaged that the compounds of the general formula I can be used in combination with a second antibiotic compound in order to provide a more efficient treatment of infections (e.g. bacterial infections) as those mentioned above. Thus, pharmaceutical compositions comprising a compound of the general formula I and a second antibiotic compound are also envisaged within the scope of the present invention.

### Novel Compounds

It is believed that almost all of the thiourea compounds defined above represent novel compounds. Hence, the present invention further provides a thiourea compound of Formula I as defined herein, which—however—is not one selected from the group consisting of:
1-Benzyl-3-(3,5-bis-trifluoromethyl-phenyl)-thiourea,
1-(3,5-Bis-trifluoromethyl-phenyl)-3-(2-chloro-benzyl)-thiourea,
1-(3,5-Bis-trifluoromethyl-phenyl)-3-(4-methyl-benzyl)-thiourea,
1-(3,5-Bis-trifluoromethyl-phenyl)-3-(4-methoxy-benzyl)-thiourea,
1-(3,5-Bis-trifluoromethyl-phenyl)-3-(4-chloro-benzyl)-thiourea,
1-(3,5-Bis-trifluoromethyl-phenyl)-3-(4-fluoro-benzyl)-thiourea,
1-(3,5-Bis-trifluoromethyl-phenyl)-3-(3,4-dichloro-benzyl)-thiourea
1-(3,5-Bis-trifluoromethyl-phenyl)-3-(3,4-dimethyl-benzyl)-thiourea,
1-(3,5-Bis-trifluoromethyl-phenyl)-3-(1-phenyl-ethyl)-thiourea,
1-(3,5-Bis-trifluoromethyl-phenyl)-3-[cyclopropyl-(4-methoxy-phenyl)-methyl]-thiourea,
1-(3,5-Bis-trifluoromethyl-phenyl)-3-phenethyl-thiourea,
1-(3,5-Bis-trifluoromethyl-phenyl)-3-[2-(4-chloro-phenyl)-ethyl]-thiourea,
1-(3,5-Bis-trifluoromethyl-phenyl)-3-[2-(3,4-dimethoxy-phenyl)-ethyl]-thiourea, and
1-(3,5-Bis-trifluoromethyl-phenyl)-3-(2-hydroxy-1-methyl-2-phenyl-ethyl)-thiourea.

Moreover, the invention also provides a pharmaceutical composition comprising a novel compound as defined above in combination with a pharmaceutically acceptable carrier.

Still further, the invention provides a novel compound as defined above for use as a drug substance.

### Formulation of Pharmaceutical Compositions

The thiourea compounds are typically formulated in a pharmaceutical composition prior to use as a drug substance.

The administration route of the compounds as defined herein may be any suitable route which leads to a concentration in the blood or tissue corresponding to a therapeutic effective concentration. Thus, e.g., the following administration routes may be applicable although the invention is not limited thereto: the oral route, the parenteral route, the cutaneous route, the nasal route, the rectal route, the vaginal route and the ocular route. It should be clear to a person skilled in the art that the administration route is dependent on the particular compound in question; particularly the choice of administration route depends on the physico-chemical properties of the compound together with the age and weight of the patient and on the particular disease or condition and the severity of the same.

The compounds as defined herein may be contained in any appropriate amount in a pharmaceutical composition, and are generally contained in an amount of about 1-95% by weight of the total weight of the composition. The composition may be presented in a dosage form which is suitable for the oral, parenteral, rectal, cutaneous, nasal, vaginal and/or ocular administration route. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, aerosols and in other suitable form.

The pharmaceutical compositions may be formulated according to conventional pharmaceutical practice, see, e.g., “Remington's Pharmaceutical Sciences” and “Encyclopedia of Pharmaceutical Technology”, edited by Swarbrick, J. & J. C. Boylan, Marcel Dekker, Inc., New York, 1988. Typically, the compounds defined herein are formulated with (at least) a pharmaceutically acceptable carrier or excipient. Pharmaceutically acceptable carriers or excipients are those known by the person skilled in the art.

Thus, the present invention provides in a further aspect a pharmaceutical composition comprising a compound of the general formula I in combination with a pharmaceutically acceptable carrier.

Pharmaceutical compositions according to the present invention may be formulated to release the active compound substantially immediately upon administration or at any substantially predetermined time or time period after administration. The latter type of compositions is generally known as controlled release formulations.

In the present context, the term “controlled release formulation” embraces i) formulations which create a substantially constant concentration of the drug within the body over an extended period of time, ii) formulations which after a predetermined lag time create a substantially constant concentration of the drug within the body over an extended period of time, iii) formulations which sustain drug action during a predetermined time period by maintaining a relatively, constant, effective drug level in the body with concomitant minimization of undesirable side effects associated with fluctuations in the plasma level of the active drug substance (saw-tooth kinetic pattern), iv) formulations which attempt to localize drug action by, e.g., spatial placement of a controlled release composition adjacent to or in the diseased tissue or organ, v) formulations which attempt to target drug action by using carriers or chemical derivatives to deliver the drug to a particular target cell type.

Controlled release formulations may also be denoted “sustained release”, “prolonged release”, “programmed release”, “time release”, “rate-controlled” and/or “targeted release” formulations.

Controlled release pharmaceutical compositions may be presented in any suitable dosage forms, especially in dosage forms intended for oral, parenteral, cutaneous nasal, rectal, vaginal and/or ocular administration. Examples include single or multiple unit tablet or capsule compositions, oil solutions, suspensions, emulsions, microcapsules, microspheres, nanoparticles, liposomes, delivery devices such as those intended for oral, parenteral, cutaneous, nasal, vaginal or ocular use.

Preparation of Solid Dosage Forms for Oral Use, Controlled Release Oral Dosage forms, fluid liquid compositions, parenteral compositions, controlled release parenteral compositions, rectal compositions, nasal compositions, percutaneous and topical compositions, controlled release percutaneous and topical compositions, and compositions for administration to the eye can be performed essentially as described in the applicant's earlier International application No. WO 99/00114, page 29, line 9, to page 40, line 3. Also, and more generally, the formulation and preparation of the above-mentioned compositions are well-known to those skilled in the art of pharmaceutical formulation. Specific formulations can be found in “Remington's Pharmaceutical Sciences”.

### Dosages

The compound are preferably administered in an amount of about 0.1-50 mg per kg body weight per day, such as about 0.5-25 mg per kg body weight per day.

For compositions adapted for oral administration for systemic use, the dosage is normally 2 mg to 1 g per dose administered 1-4 times daily for 1 week to 12 months depending on the disease to be treated.

The dosage for oral administration for the treatment of bacterial diseases is normally 1 mg to 1 g per dose administered 1-4 times daily for 1 week to 12 months; in particular, the treatment of tuberculosis will normally be carried out for 6-12 months.

The dosage for oral administration of the composition in order to prevent diseases is normally 1 mg to 75 mg per kg body weight per day. The dosage may be administered once or twice daily for a period starting 1 week before the exposure to the disease until 4 weeks after the exposure.

For compositions adapted for rectal use for preventing diseases, a somewhat higher amount of the compound is usually preferred, i.e. from approximately 1 mg to 100 mg per kg body weight per day.

For parenteral administration, a dose of about 0.1 mg to about 50 mg per kg body weight per day is convenient. For intravenous administration, a dose of about 0.1 mg to about 20 mg per kg body weight per day administered for 1 day to 3 months is convenient. For intraarticular administration, a dose of about 0.1 mg to about 20 mg per kg body weight per day is usually preferable. For parenteral administration in general, a solution in an aqueous medium of 0.5-2% or more of the active ingredients may be employed.

For topical administration on the skin, a dose of about 1 mg to about 5 g administered 1-10 times daily for 1 week to 12 months is usually preferable.

In many cases, it will be preferred to administer the compound defined herein together with another antibiotic drug, thereby reducing the risk of development of resistance against the conventional drugs, and reducing the amount of each of the drugs to be administered, thus reducing the risk of side effects caused by the conventional drugs.

### Screening

In a further aspect, the invention further provides combinatorial libraries, mixtures and kits for screening compounds as defined above.

In one embodiment, a combinatorial library comprising at least two compounds of the general formula I is provided. Such library may be in the form of an equimolar mixture, or in a mixture of any stoichiometry. Typical embodiments comprise at least two, such as at least 10, such as at least 100, such as at least 1000, such as at least 10,000, such as at least 100,000 compounds as defined above.

In another embodiment, combinatorial compound collections in the form of kits for screening for biologically or pharmacologically active compounds are provided. Such kits comprise at least two topologically distinct singular compounds of the general formula I. Typical kits comprise at least 10, such as at least 100, such as at least 1000, such as at least 10,000, such as at least 100,000 compounds as defined above. Kits are preferably provided in the form of solutions of the compounds in appropriate solvents.

Further provided are methods for screening for pharmacologically active compounds, especially bacteriocidal agents, consisting of the steps of preparing a kit or library comprising at least two compounds of the general formula I, contacting said kit or library with a target molecule, such as a protein or nucleic acid, a target tissue, or a target organism, such as a bacterium and detecting a biological or pharmacological response caused by at least one compound. Optionally, the steps may be repeated when appropriate to achieve deconvolution.

### EXAMPLES

### Compound A—1-(3,5-Bis-trifluoromethyl-phenyl)-3-(3-chloro-benzyl)-thiourea

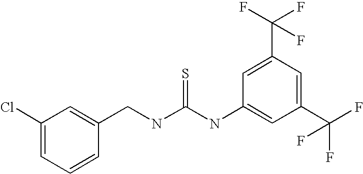

A solution of 3-chloro-benzylamine (1 mmol) and 3,5-bis-(trifluoromethyl)phenyl isothiocyanate (1 mmol) in CHCl₂ (5 mL) was stirred at r.t. for 24 h. The precipitate was collected and washed with n-heptane, giving compound A as white crystals (180 mg).

### Compound B—1-(3,5-Bis-trifluoromethyl-phenyl)-3-(2,4-dichloro-benzyl)-thiourea

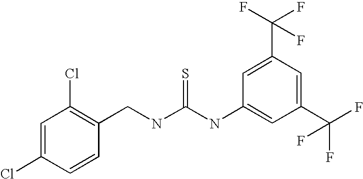

A solution of 2,4-dichloro-benzylamine (1 mmol) and 3,5-bis-(trifluoromethyl)phenyl isothiocyanate (1 mmol) in CHCl₂ (5 mL) was stirred at r.t. for 24 h. The precipitate was collected and washed with n-heptane, giving compound B as white crystals (366 mg).

### Compound C—1-(3,5-Bis-trifluoromethyl-phenyl)-3-[1-(4-bromo-phenyl)-ethyl]-thiourea

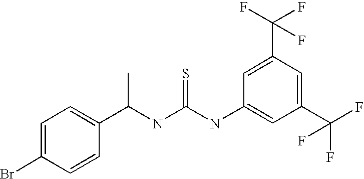

A solution of 4-bromo-alfa-methylbenzylamine (1 mmol) and 3,5-bis-(trifluoromethyl)phenyl isothiocyanate (1 mmol) in CHCl₂ (5 mL) was stirred at r.t. for 24 h. The precipitate was collected and washed with n-heptane, giving compound C as white crystals (158 mg).

### Compound D—1-(3,5-Bis-trifluoromethyl-phenyl)-3-[2-(2,4-dichloro-phenyl)-ethyl]-thiourea

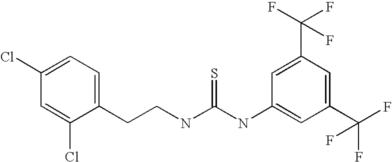

A solution of 2,4-dichloro-phenethylamine (1 mmol) and 3,5-bis-(trifluoromethyl)phenyl isothiocyanate (1 mmol) in CHCl₂ (5 mL) was stirred at r.t. for 24 h. The precipitate was collected and washed with n-heptane, giving compound D as white crystals (378 mg).

### Compound E—1-(3,5-Bis-trifluoromethyl-phenyl)-3-[2-(3,4-dichloro-phenoxy)-ethyl]-thiourea

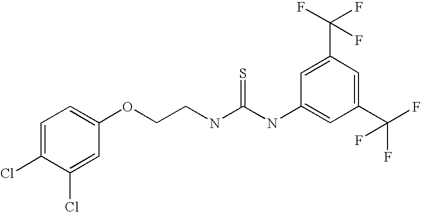

### Step 1: 2-(3,4-Dichloro-phenoxy)-ethylamine

A solution of 3,4-dichlorophenol (8.15 g, 50 mmol) in anhydrous DMF (100 mL) was cooled on ice and added sodium hydride (60%, 2.0 g, 60 mmol) in portions. The solution was stirred for 2 min and N-(2-bromoethyl)phthalimide was added. Stirring was continued for 18 h and the mixture was mixed with water (500 mL)/2N NaOH (50 mL) giving crystals which were recrystallised from acetonitrile (12 g, 71%). The crystals were suspended in ethanol (120 mL) and hydrazine hydrate (2.0 g, 42 mmol) was added. Stirring for 1 h at r.t. and 1 h at 60° C. and subsequent cooling gave, after filtration, a solution that was concentrated under vacuum. 2N NaOH (50 mL) was added and the compound was extracted with EtOAC (3×50 mL). Concentration gave the desired product as a yellow oil (4 g, 39%)

### Step 2

A solution of 2-(3,4-Dichloro-phenoxy)-ethylamine (1 mmol) and 3,5-bis-(trifluoromethyl)phenyl isothiocyanate (1 mmol) in CHCl₂ (5 mL) was stirred at r.t. for 24 h. The precipitate was collected and washed with n-heptane, giving compound E as white crystals (425 mg).

### Further Compounds

Compound F (1-(3,5-Bis-trifluoromethyl-phenyl)-3-(4-chloro-benzyl)-thiourea) and Compound G (1-(3,5-Bis-trifluoromethyl-phenyl)-3-[2-(3-chloro-phenyl)-ethyl]-thiourea) were prepared in a similar manner.

### Biological Testing

### Antibacterial Screening (MIC)

An initial screening for in vitro activity was conducted in a broth microtiter assay. The synthesized compounds were assayed against several Gram+bacteria such as Staphylococcus aureus and 2 different strains of E. coli (including a type strain and an antibiotic-susceptible strain). Following initial screening MICs were determined.

Staphylococcus aureus ATCC29213
Staphylococcus aureus ATCC33591 (MRSA)
Eschericia coli ATCC25922
Eschericia coli ESS

The screening was performed with test compounds in 4 different concentrations.

For compounds exhibiting activity in the screening assay MIC was then determined in a broth microdilution assay as described by NCLLS (M7-A5) modified to include uninoculated dilution series of test compounds to facilitate MIC determination if the test compound should precipitate. MICs for ATCC type strains fall within the limits posted by the NCCLS (M100-S11) when tested against Vancomycin, Tetracycline and Gentamycin.

### Protein Binding

Protein binding was performed using a bioassay determining MICs in the presence or absence of 40 mg/ml bovine serum albumin (BSA).

### In Vitro Cytotoxicity (Haemolysis and MTT)

The cytotoxicity effect of the compounds at different exposure times as a function of concentration has been tested in an erythrocyte hemolysis assay as well as an MTT assay on MCF7 cells (ATCC: HTB 22).

### Biological Results

| | MIC | | | | | |
|---|---|---|---|---|---|---|
| | Staph. | | Protein | Haemolysis | | |
| Compound | aureus | | binding | 4x MIC | 50% Haemolysis | MTT |
| Compound A | 0.6 | μM | 98% | <1% | 75 μM | 150 μM |
| Compound B | 0.15/0.3 | μM | >98% | <1% | 150 μM | 150 μM |
| Compound C | 0.3 | μM | 98% | 15% | 37.5 μM | ND |
| Compound D | 0.3 | μM | 98% | ND | ND | 150 μM |
| Compound E | 0.3 | μM | 98% | ND | 18.8 μM | ND |
| Compound F | 0.3/0.6 | μM | >97% | <1% | 150 μM | 150 μM |
| Compound G | 0.6 | μM | >97% | 0% | 75 μM | 150 μM |
| Ciprofloxicin | 1.5 | μM | ND | 0% | 150 μM | >100 μM |
| Dicloxacillin | 0.5 | μM | ND | 0% | 150 μM | >100 μM |
| Vancomycin | 0.3 | μM | ND | 0% | 150 μM | >100 μM |

ND: Not done.

The invention claimed is:

1. Use of a thiourea compound for the preparation of a pharmaceutical composition for the treatment of an infection, said compound having the Formula I 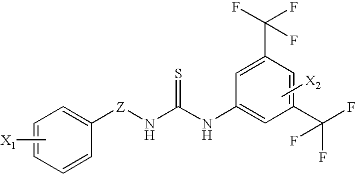
wherein
X¹ designate a substituent present 0-5 times on the respective benzene ring and X² designate a substituent present 0-3 times on the respective benzene ring, each X¹ and X² independently being selected from the group consisting of optionally substituted C₁₋₁₂-alkyl, optionally substituted C₂₋₁₂-alkenyl, optionally substituted C₄₋₁₂-alkadienyl, optionally substituted C₆₋₁₂-alkatrienyl, optionally substituted C₂₋₁₂-alkynyl, hydroxy, optionally substituted C₁₋₁₂-alkoxy, optionally substituted C₂₋₁₂-alkenyloxy, carboxy, optionally substituted C₁₋₁₂-alkoxycarbonyl, optionally substituted C₁₋₁₂-alkylcarbonyl, formyl, C₁₋₆-alkylsulphonylamino, optionally substituted aryl, optionally substituted aryloxycarbonyl, optionally substituted aryloxy, optionally substituted arylcarbonyl, optionally substituted arylamino, arylsulphonylamino, optionally substituted heteroaryl, optionally substituted heteroaryloxycarbonyl, optionally substituted heteroaryloxy, optionally substituted heteroarylcarbonyl, optionally substituted heteroarylamino, heteroarylsulphonylamino, optionally substituted heterocyclyl, optionally substituted heterocyclyloxycarbonyl, optionally substituted heterocyclyloxy, optionally substituted heterocyclylcarbonyl, optionally substituted heterocyclylamino, heterocyclylsulphonylamino, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, amino-C₁₋₆-alkyl-carbonylamino, mono- and di(C₁₋₆-alkyl)amino-C₁₋₆-alkyl-carbonylamino, cyano, guanidino, carbamido, C₁₋₆-alkanoyloxy, C₁₋₆-alkylsulphonyl, C₁₋₆-alkylsulphinyl, C₁₋₆-alkylsulphonyloxy, aminosulfonyl, mono- and di(C₁₋₆-alkyl)aminosulfonyl, nitro, optionally substituted C₁₋₆-alkylthio, and halogen, where any nitrogen-bound C₁₋₆-alkyl is optionally substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino; or two X¹ substituents may together form an —OCH(R¹)O— group wherein R¹ is selected from the group consisting of hydrogen, C₁₋₆-alkyl and phenyl;
wherein Z is selected from the group consisting of —(CHR)—, —(CHR)₂—, —O—(CHR)₂—, —NH—(CHR)₂—, and —S—(CHR)₂—, wherein each R individually is selected from the group consisting of hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, optionally substituted C₁₋₆-alkylcarbonyl, optionally substituted aryl, optionally substituted aryloxy, optionally substituted arylcarbonyl, optionally substituted heteroaryl, optionally substituted heteroaryloxy, optionally substituted heteroarylcarbonyl, optionally substituted heterocyclyl, optionally substituted heterocyclyloxy, optionally substituted heterocyclylcarbonyl; including salts thereof.

2. The use according to claim 1, wherein R is selected from the group consisting of hydrogen and C₁₋₆-alkyl.

3. The use according to claim 1, wherein X¹ and X² independently designates 0-4 substituents, where such optional substituents independently are selected from optionally substituted C₁₋₁₂-alkyl, hydroxy, optionally substituted C₁₋₁₂-alkoxy, optionally substituted C₂₋₁₂-alkenyloxy, carboxy, optionally substituted C₁₋₁₂-alkylcarbonyl, formyl, C₁₋₆-alkylsulphonylamino, optionally substituted aryl, optionally substituted aryloxy-carbonyl, optionally substituted aryloxy, optionally substituted arylcarbonyl, optionally substituted arylamino, arylsulphonylamino, optionally substituted heteroaryl, optionally substituted heteroarylamino, optionally substituted heteroarylcarbonyl, optionally substituted heteroaryloxy, heteroarylsulphonylamino, optionally substituted heterocyclyl, optionally substituted heterocyclyloxy, optionally substituted heterocyclylamino, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, amino-C₁₋₆-alkyl-carbonylamino, mono- and di(C₁₋₆-alkyl)amino-C₁₋₆-alkyl-carbonylamino, guanidino, carbamido, C₁₋₆-alkylsulphonyl, C₁₋₆-alkylsulphinyl, C₁₋₆-alkylsulphonyloxy, optionally substituted C₁₋₆-alkylthio, aminosulfonyl, mono- and di(C₁₋₆-alkyl)aminosulfonyl, and halogen, where any nitrogen-bound C₁₋₆-alkyl may be substituted with a substituent selected from the group consisting of hydroxy, C₁₋₆-alkoxy, and halogen.

4. The use according to claim 1, wherein X¹ and X² independently designate 0-3 substituents, such optional substituents independently being selected from optionally substituted C₁₋₆-alkyl, optionally substituted C₁₋₆-alkoxy, optionally substituted C₁₋₆-alkylcarbonyl, optionally substituted aryl, optionally substituted aryloxy, optionally substituted arylamino, optionally substituted heteroaryl, optionally substituted heteroarylamino, mono- and di(C₁₋₆-alkyl)amino, C₁₋₆-alkylcarbonylamino, optionally substituted C₁₋₆-alkylthio, optionally substituted heterocyclyl, optionally substituted heterocyclyloxy, optionally substituted heterocyclylamino and halogen, where any nitrogen-bound C₁₋₆-alkyl may be substituted with a substituent selected from the group consisting of hydroxy, C₁₋₆-alkoxy, and halogen.

5. The use according to claim 1, wherein Z is —(CHR)— where R is selected from hydrogen and C₁₋₆-alkyl.

6. The use according to claim 1, wherein X¹ represents at least one substituent selected from C₁₋₆-alkyl, C₁₋₆-alkoxy, C₁₋₆-alkylcarbonyl, optionally substituted aryl, optionally substituted aryloxy, optionally substituted arylamino, optionally substituted heteroaryl, optionally substituted heteroarylamino, mono- and di(C₁₋₆-alkyl)amino, C₁₋₆-alkylcarbonylamino, optionally substituted C₁₋₆-alkylthio, optionally substituted heterocyclyl, optionally substituted heterocyclyloxy, optionally substituted heterocyclylamino, and halogen.

7. The use according to claim 1, wherein no X² substituents are present.

8. The use according to claim 1, wherein X¹ represents 1-3 substituents selected from C₁₋₆-alkyl, C₁₋₆-alkoxy, and halogen.

9. The use according to claim 1, wherein X¹ at least one halogen substituent.

10. The use according to claim 1, wherein the compound of Formula I is one of:

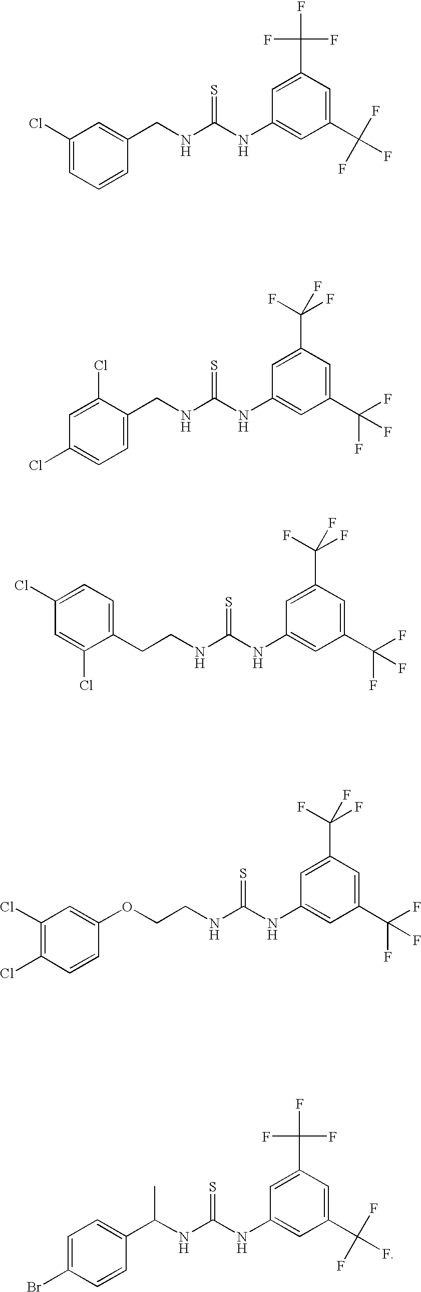

11. The use according to claim 1, wherein the infection is associated with bacteria, fungi, mycoplasma or other microorganisms.

12. The use according to claim 11, wherein the infection is associated with bacteria selected from the group consisting of Gram-positive bacteria, Gram-negative bacteria, microaerophilic bacteria and anaerobic bacteria.

13. The use according to claim 12, wherein the bacteria is selected from antibiotic-sensitive and -resistant strains of S. aureus.

14. The use according to claim 12, wherein the bacteria is a member of Enterobacteriaceae.

15. The use according to claim 11, wherein the infection is associated with fungi.

16. The use according to claim 11, wherein the infection is associated with mycoplasma.

17. A thiourea compound of Formula I as defined in the claim 1, which is not one selected from the group consisting of:
1-Benzyl-3-(3,5-bis-trifluoromethyl-phenyl)-thiourea,
1-(3,5-Bis-trifluoromethyl-phenyl)-3-(2-chloro-benzyl)-thiourea,
1-(3,5-Bis-trifluoromethyl-phenyl)-3-(4-methyl-benzyl)-thiourea,
1-(3,5-Bis-trifluoromethyl-phenyl)-3-(4-methoxy-benzyl)-thiourea,
1-(3,5-Bis-trifluoromethyl-phenyl)-3-(4-chloro-benzyl)-thiourea,
1-(3,5-Bis-trifluoromethyl-phenyl)-3-(4-fluoro-benzyl)-thiourea,
1-(3,5-Bis-trifluoromethyl-phenyl)-3-(3,4-dichloro-benzyl)-thiourea,
1-(3,5-Bis-trifluoromethyl-phenyl)-3-(3,4-dimethyl-benzyl)-thiourea,
1-(3,5-Bis-trifluoromethyl-phenyl)-3-(1-phenyl-ethyl)-thiourea,
1-(3,5-Bis-trifluoromethyl-phenyl)-3-[cyclopropyl-(4-methoxy-phenyl)-methyl]-thiourea,
1-(3,5-Bis-trifluoromethyl-phenyl)-3-phenethyl-thiourea,
1-(3,5-Bis-trifluoromethyl-phenyl)-3-[2-(4-chloro-phenyl)-ethyl]-thiourea,
1-(3,5-Bis-trifluoromethyl-phenyl)-3-[2-(3,4-dimethoxy-phenyl)-ethyl]-thiourea, and
1-(3,5-Bis-trifluoromethyl-phenyl)-3-(2-hydroxy-1-methyl-2-phenyl-ethyl)-thiourea.

18. A pharmaceutical composition comprising a compound as defined in claim 17 in combination with a pharmaceutically acceptable carrier.

19. The compound as defined in claim 17 for use as a drug substance.

20. A method of treating a mammal suffering from an infection, said method comprising the step of administering a therapeutically effective amount of a thiourea compound of Formula I as defined in claim 1 to said mammal.

21. The method according to claim 20, wherein the infection is associated with bacteria, fungi, mycoplasma or other microorganisms.

22. The method according to claim 21, wherein the bacterial infection is associated with bacteria selected from Gram-positive bacteria, Gram-negative bacteria, microaerophilic bacteria and anaerobic bacteria.

23. The method according to claim 22, wherein the bacteria is selected from antibiotic-sensitive and -resistant strains of S. aureus.

24. The method according to claim 22, wherein the bacteria is a member of Enterobacteriaceae.

25. The method according to claim 21, wherein the infection is associated with fungi.

26. The method according to claim 21, wherein the infection is associated with mycoplasma.
